(19) United States
(12) Patent Application Publication
(10) Pub. No.: US 2009/0286884 A1
(43) Pub. Date: Nov. 19, 2009

(54) Hops Beta-Acid Anti-Diabetic Composition

(75) Inventors: Mitsunori Ono, Lexington, MA (US); John Paul Maye, Washington, DC (US)

Correspondence Address: EDWARDS ANGELL PALMER & DODGE LLP, P.O. BOX 55874, BOSTON, MA, 02205, US

(73) Assignee: John I. Haas, Washington, DC (US)

(21) Appl. No.: 12/372,614

(22) Filed: Feb. 17, 2009

Related U.S. Application Data
(63) Continuation of application No. 11/791,430.
(60) Provisional application No. 60/630,232, filed on Nov. 22, 2004.

(51) Int. Cl. A61K 31/12

(52) U.S. Cl. 514/689

(57) ABSTRACT

The invention relates to compositions and processes for reducing the level of blood glucose or ameliorating diabetic symptoms in a subject by treatment with the composition which includes one or more safe and suitable hops acids or hops acid derivatives. More specifically, the process comprises using an ingredient or applying to a food or a nonfood product a composition-comprising beta hops acids in order to reduce blood glucose levels in Type 2 diabetics or its related diseases.

### BACKGROUND

Diabetes mellitus is the most common metabolic disease worldwide. Every day, 1700 new cases of diabetes are diagnosed in the United States, and at least one-third of the 16 million Americans with diabetes are unaware of it. Diabetes is the leading cause of blindness, renal failure, and lower limb amputations in adults and is a major risk factor for cardiovascular disease and stroke.

Normal glucose homeostasis requires the finely tuned orchestration of insulin secretion by pancreatic beta cells in response to subtle changes in blood glucose levels, delicately balanced with secretion of counter-regulatory hormones such as glucagon. Type 1 diabetes results from autoimmune destruction of pancreatic beta cells causing insulin deficiency. Type 2 or noninsulin-dependent diabetes mellitus (NIDDM) accounts for >90% of cases and is characterized by a triad of (1) resistance to insulin action on glucose uptake in peripheral tissues, especially skeletal muscle and adipocytes, (2) impaired insulin action to inhibit hepatic glucose production, and (3) dysregulated insulin secretion (DeFronzo, (1997) Diabetes Rev. 5:177-269). In most cases, type 2 diabetes is a polygenic disease with complex inheritance patterns (reviewed in Kahn et al., (1996) Annu. Rev. Med. 47:509-531).

Environmental factors, especially diet, physical activity, and age, interact with genetic predisposition to affect disease prevalence. Susceptibility to both insulin resistance and insulin secretory defects appears to be genetically determined (Kahn, et al). Defects in insulin action precede the overt disease and are seen in nondiabetic relatives of diabetic subjects. In spite of intense investigation, the genes responsible for the common forms of Type 2 diabetes remain unknown.

One of the fundamental actions of insulin is to stimulate uptake of glucose from the blood into tissues, especially muscle and fat. This occurs via facilitated diffusion, which is mediated by specific glucose transporter proteins that insert into the plasma membrane of cells. GLUT4 is the most important insulin-sensitive glucose transporter in these tissues. Insulin binds to its receptor in the plasma membrane, generating a series of signals that result in the translocation or movement of GLUT4 transporter vesicles to the plasma membrane, where a first docking step, followed by fusion with the plasma membrane takes place; after an activation or exposure step takes place, glucose enters the cell. Studies in both animals and humans indicate that alterations in GLUT4 expression, trafficking, and/or activity occur in adipose cells and muscle in diabetes and other insulin-resistant states (Abel et al., Diabetes Mellitus: A Fundamental and Clinical Text (1996) pp. 530-543.). New and innovative treatments for diabetes are clearly a priority for researchers in this field. It is known that several hops ingredients reduce insulin resistance and improve insulin sensitivity in high fat-fed mice with insulin resistance and in patients with type 2 diabetes. (J Biol. Chem. 2004, 279: 33456-33462). However, the reported active ingredients from hops are limited to two major isohumulone homologs, isohumulone and isocohumulone. The two active ingredients are categorized into hops alpha acids, characterized as having two isoprene units (with or without double bonds), each bonded to one of two different carbon atoms that are part of a core six membered ring structure in those compounds. In addition, it is demonstrated that the improvement of insulin sensitivity is observed through PPARgamma activation.

### SUMMARY

The invention relates to heterocyclic compounds, compositions comprising the compounds, and methods of using the compounds and compound compositions. The compounds and compositions comprising them are useful for treating disease or disease symptoms, including those mediated by or associated with glucose and glucose level regulation.

One aspect is a composition having at least one compound selected from hops beta acids, hops beta acid derivatives, and their use to provide a method of treating diabetes and related diseases, such as obesity, by administering to a subject the aforementioned compositions. The compounds described herein are glucose uptake enhancers.

One embodiment of the present invention is an anti-diabetic composition comprising a component having at least one compound that is a hops beta acid or hops beta acid derivative. Particularly preferred is a composition including (e.g., comprising, consisting essentially of, consisting of) one or more of Lupulone, Adlupulone or Colupulone (e.g., combinations of any two or a combination of all three). In other aspects, the combination of Lupulone, Adlupulone or Colupulone is about 85%, about 90%, about 95%, or about 98% of the beta-acids in the entire composition. In other aspects, each of Colupulone, Lupulone, or Adlupulone is about 65%, about 70%, about 75%, about 80%, about 85%, about 90% or about 95% of the beta acid(s) in the entire composition. In one aspect, the ratio of Colupulone, Lupulone, and Adlupulone is about 65:25:10, respectively.

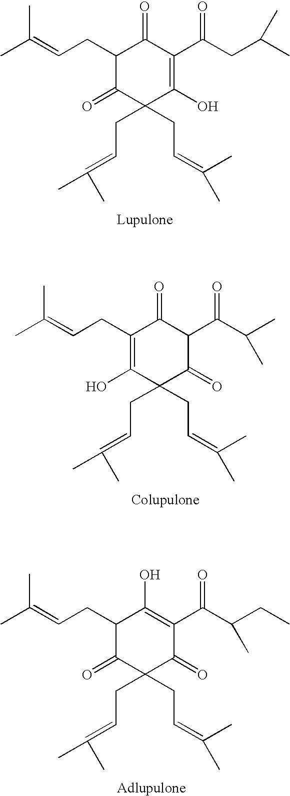

An alternate embodiment is that wherein the hops beta acid or hops beta acid derivative is one or more isolated compounds. More specifically, an isolated beta hops acid, or isolated Lupulone, Adlupulone or Colupulone. A hops beta acid is a naturally occurring molecule extracted from hops that includes two isoprene units (with or without double bonds, e.g., isoprenyl or isopentyl groups) attached to the same carbon (e.g., geminal substituted) that is part of a five or six membered core ring structure (e.g., a cyclopentyl or cyclohexyl ring). Representative examples include:

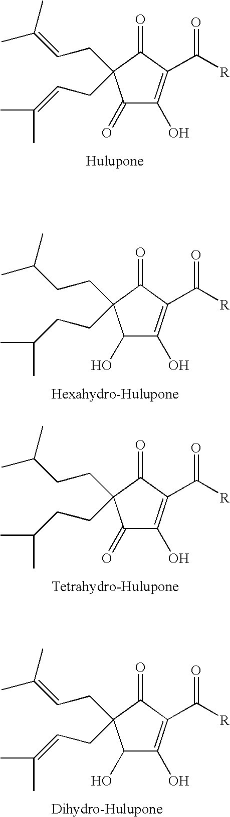

where each R is independently alkyl, alkenyl, cycloalkylalkyl, or aralkyl

The hops acid or hops acid derivative can be of formula (I):

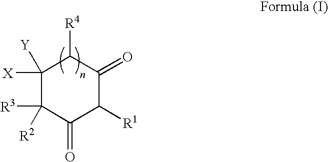

or pharmaceutically acceptable salt, solvate, hydrate, or prodrug thereof,

wherein

each R¹ is independently —C(O)R⁵;

each R² is independently alkyl or alkenyl;

each R³ is independently alkyl or alkenyl each R⁴ is independently alkyl or alkenyl;

each R⁵ is independently alkyl, alkenyl, cycloalkylalkyl, or aralkyl;

each X is independently H, OH or taken together with Y is O;

each Y is independently H, OH or taken together with X is O; and

each n is independently 0 or 1.

In one aspect, the compounds are those of any of the formulae herein, wherein X and Y are not the same; wherein X and Y are the same; wherein X and Y taken together are O (e.g., X, Y and the carbon to which they are both attached form a carbonyl); wherein one of X and Y is H and the other is OH.

A hops acid derivative is a compound that is chemically derived (either through natural biosynthetic procesess (e.g., living organism metabolism (e.g., mammal, plant, bacetria)) or synthetic processes using human intervention (e.g., chemical synthesis)) from a hops acid. A beta-hops acid derivative is a compound derived from a beta-hops acid.

Another aspect is a composition having a hops acids or hops acid derivative and a pharmaceutically acceptable carrier. The composition can further have an additional therapeutic agent. The compositions herein also include nutraceutical compositions comprising one or more of the aforementioned compound(s) and a nutraceutically acceptable carrier. The composition can include one or more additional nutraceutical agents.

Additional anti-diabetic agents can be one or more selected from: metformin, a biguanide, a sulfonyl urea, a glucosidase inhibitor, a PPAR .gamma. agonist, a PPAR .alpha./.gamma. dual agonist, an aP2 inhibitor, DPP-IV (dipeptidyl peptidase IV), DPP-IV inhibitor, LAF-237, BMS-477188, MK-0431, GSK-23A, a DP4 inhibitor, an insulin sensitizer, a glucagon-like peptide-1 (GLP-1), GLP-1 agonist, exendin-4, liraglutide, insulin, a meglitinide, a PTP1B inhibitor, a glycogen phosphorylase inhibitor, or a glucoser-6-phosphatase inhibitor, for example.

Another aspect is a method of treating a disease or disease symptom in a subject in need of such treatment (including identified as in need of such treatment) comprising administering to the subject an effective amount of a compound of any of the compounds herein. The method can be wherein the disease or disease symptom is modulated (e.g., inhibiting, agonism, antagonism) by glucose or glucose level regulation. The disease or disease symptom can be diabetes, diabetic retinopathy, diabetic neuropathy, diabetic nephropathy, delayed wound healing, insulin resistance, hyperglycemia, hyperinsulinemia, elevated blood levels of fatty acids or glycerol, hyperlipidemia, obesity, hypertriglyceridemia, Syndrome X, diabetic complications, atherosclerosis or hypertension.

In other aspects, the invention relates to a composition comprising a compound of any of the formulae herein, an additional therapeutic agent, and a pharmaceutically acceptable carrier. The additional therapeutic agent can be an anti-diabetic or an anti-obesity agent (e.g., Meridia®, phentermine, Tenuate®, Xenical®).

Yet another aspect of this invention relates to a method of treating a subject (e.g., mammal, human, horse, dog, cat) having or suffering from a disease or disease symptom (including, but not limited to, diabetes mellitus, diabetes, diabetic retinopathy, diabetic neuropathy, diabetic nephropathy, delayed wound healing, insulin resistance, hyperglycemia, hyperinsulinemia, elevated blood levels of fatty acids or glycerol, hyperlipidemia, obesity, hypertriglyceridemia, Syndrome X, diabetic complications, atherosclerosis or hypertension). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g., opinion) or objective (e.g., measurable by a test or diagnostic method).

Yet another aspect of this invention relates to a method of treating a subject (e.g., mammal, human, horse, dog, cat) having a glucose-mediated disease or disease symptom (including, but not limited to diabetes mellitus, diabetes, type-II diabetes, diabetic retinopathy, diabetic neuropathy, diabetic nephropathy, delayed wound healing, insulin resistance, hyperglycemia, hyperinsulinemia, elevated blood levels of fatty acids or glycerol, hyperlipidemia, obesity, hypertriglyceridemia, Syndrome X, diabetic complications, atherosclerosis or hypertension). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g., opinion) or objective (e.g., measurable by a test or diagnostic method). The methods herein are also those wherein the subject is, in fact, treated, as shown by diagnostic test or opinion of subject or health care provider.

The methods also include a method of reducing blood-glucose levels in a subject including administering to the subject (including a subject identified as in need of such treatment) an effective amount of a compound described herein, or a composition described herein to produce such effect. The methods also include a method of modulating GLUT4 expression levels in a subject including administering to the subject (including a subject identified as in need of such treatment) an effective amount of a compound described herein, or a composition described herein to produce such effect. The methods can include the steps of monitoring (assay, diagnostic test) glucose levels either prior to, subsequent to, or both, administration of the compounds or compositions herein.

In another aspect, the composition comprises a component having at least one compound that is a hops beta acid or hops beta acid derivative and a compound found in turmeric (e.g., curcumin). Particularly, a composition including (e.g., comprising, consisting essentially of, consisting of) one or more of Lupulone, Adlupulone or Colupulone (e.g., combinations of any two or a combination of all three) and a compound found in turmeric (e.g., curcumin). It is surprisingly found that these combinations have synergistic effects on enhancement of glucose uptake. See, FIG. 4. That is, the combination composition shows enhanced glucose uptake relative to either of the beta-acid mixture or curcumin alone.

The invention also relates to a method of making a compound described herein, the method including any reactions or reagents or processes (including extraction, isolation, purification) as delineated in the schemes or examples herein. Alternatively, the method includes taking any one of the intermediate compounds described herein and reacting it with one or more chemical reagents in one or more steps to produce a compound described herein.

Also within the scope of this invention is a packaged product. The packaged product includes a container, one (or more) of the aforementioned compounds in the container, and a legend (e.g., a label or an insert) associated with the container and indicating administration of the compound for treating a disorder associated with glucose level modulation.

In other embodiments, the compounds, compositions, and methods delineated herein are any of the compounds delineated herein or methods including them.

The details of one or more embodiments of the invention are set forth in the accompanying drawings and the description below. Other features, objects, and advantages of the invention will be apparent from the description and from the claims.

### BRIEF DESCRIPTION OF THE DRAWINGS

FIG. 1 shows the enhancing effect of ³H-deoxyglucose uptake into skeletal muscle cells after 24 hrs with various hops extracts.

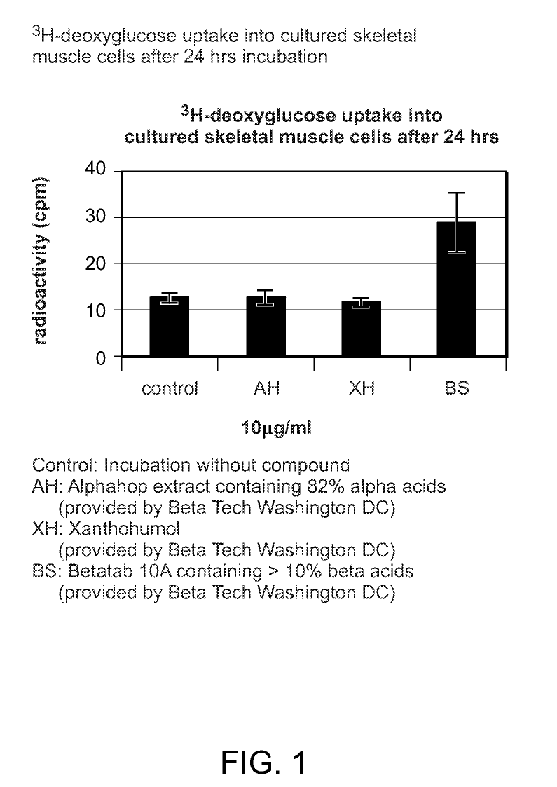

FIG. 2 shows 3H-deoxyglucose uptake into skeletal muscle cells in the presence of several hops acids.

FIG. 3 shows a timecourse of 3H-deoxyglucose uptake into skeletal muscle cells with beta hops acids.

FIG. 4 shows the synergistic effects of 3H-deoxyglucose uptake into skeletal muscle cells of the combination of curcumin (98% from Sigma) with beta hops acids (e.g., Lupulone, Adlupulone or Colupulone is about 65:25:10, respectively) relative to either alone.

### DETAILED DESCRIPTION

The present invention provides a novel anti-diabetic composition and its use in a process of reducing blood glucose levels in a subject. The novel anti-diabetic composition comprises one or more hops acids or hops acid derivatives.

The component of the novel composition is one or more hops acids or acid derivatives. The bitter acids component of the hops used in beer making and the most prevalent groups of bitter acids found as components of hops are the alpha-acids and the beta acids, also referred to as humulones and lupulones, respectively. Both contribute bitterness to beer, but the alpha-acids are much more intense in this regard than the beta acids. However, test results indicate that neither alpha-acid nor Xanthohumol, which is known as a strong antibacterial ingredient in hops, show any enhancing effect under these conditions.

Hops contain two major organic acid classes, Humulones (also known as alpha acids) and Lupulones (also known as beta acids). There are believed to be many analogs of alpha acids and beta acids, however, there are three major analogs for alpha acids and three major analogs for beta acids. These respective groups of three major analogs make-up roughly 99% or more of the alpha acids and beta acids, respectively. These three major analogs are also the ones generally discussed in the literature. The three major analogs for alpha acids and beta acids along with their general percentages are:

Alpha Acids (Humulones): co-humulone (25-45%), n-humulone(25-65%), ad-humulone(10-15%).

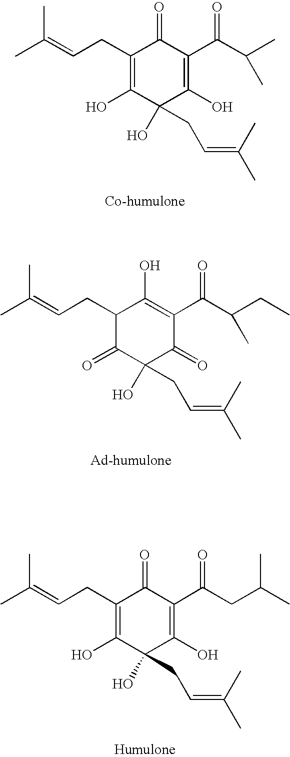

Beta Acids (Lupulones): co-lupulone(50-65%), lupulone(25-35%) and ad-lupulone (10-15%). (Structures above).
Because differences in hop varieties, the percent co-humulone for example can be as low as 25% or as high as 45%, however, if one looks at a specific hop variety, there is generally consistency in the percentage of co-humulone, for example, in that particular hop variety. Representative varieties include the following:

| | | |
|---|---|---|
| Name | Maturity Major | Growing Area(s) |

Aroma Varieties

| | | |
|---|---|---|
| Cascade | Medium | WA/OR/ID |
| Fuggle | Early | WA/OR |
| Golding | Medium | WA/OR |
| Mt. Hood | Medium | WA/OR |
| Tettnanger | Early | WA/OR/ID |
| Willamette | Medium | WA/OR/ID |

Bitter Varieties

| | | |
|---|---|---|
| Chelan | Medium | WA |
| Chinook | Medium | WA/ID |
| Cluster | Early/Late | WA/ID |
| Galena | Medium | WA/ID |
| Millennium | Late | WA/OR |
| Nugget | Late | WA/OR |
| Super High Alpha | Late | WA |
| Tillicum | Early | WA |

Key: ID = Idaho, OR = Oregon, WA = Washington State

As used herein, the term “halo” refers to any radical of fluorine, chlorine, bromine or iodine.

The term “alkyl” refers to a hydrocarbon chain that may be a straight chain or branched chain, containing 1-20 or the indicated number of carbon atoms. For example, C₁-C₅ indicates that the group may have from 1 to 5 (inclusive) carbon atoms in it. The term “lower alkyl” refers to a C₁-C₆ alkyl chain. The term “alkenyl” refers to a hydrocarbon chain that may be a straight chain or branched chain, containing 1-20 or the indicated number of carbon atoms and one or more double bonds in the chain (e.g., propylenyl, isopentylenyl). For example, C₁-C₁₀ indicates that the group may have from 1 to 10 (inclusive) carbon atoms in it. The term “arylalkyl” refers to a moiety in which an alkyl hydrogen atom is replaced by an aryl group. The term “cycloalkylalkyl” refers to a moiety in which an alkyl hydrogen atom is replaced by a cycloalkyl group.

The term “aryl” refers to an aromatic monocyclic, bicyclic, or tricyclic ring system having carbon ring atoms, wherein 0, 1, 2, 3, or 4 atoms of each ring may be substituted by a substituent.

The term “cycloalkyl” as employed herein includes saturated and partially unsaturated cyclic hydrocarbon groups having 3 to 12 carbons, preferably 3 to 8 carbons, and more preferably 3 to 6 carbons.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg. Effective doses will also vary depending on route of administration, as well as the possibility of co-usage with other agents.

A “anti-diabetic” or “hypoglycemic” compound or composition refers to an agent that lowers blood glucose levels. The hypoglycemic or anti-diabetic effect can be measured by a variety of methods including, but not limited to, measuring the blood glucose levels, the rate of insulin binding to its receptor, the level of insulin secretion from pancreatic beta cells, and inhibition of glucohydrolase activity. Such methods are known in the art.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Beta-acids can be prepared by purification from natural hops and also chemical synthesis according to traditional methods. The compounds delineated herein can be synthesized using conventional methods known in the art.

The term “extract” refers to a concentrated preparation of the essential constituents of a plant (e.g., medicinal plant, hops). Typically, an extract is prepared by drying and powderizing the plant. Optionally, the plant, the dried plant or the powderized plant may be boiled in solution. The extract may be used in liquid form, or it may be mixed with other liquid or solid herbal extracts. Alternatively, the herbal extract may be obtained by further precipitating solid extracts from the liquid form. Edible plant extracts include those from any plant that is edible to a human (e.g., fruit extract, vegetable extract, root extract, leaf extract, tree or bark extract, bean extract, and the like) and includes, for example, green tea extract, red onion extract, grape seed extract, cocoa extract, red clover extracts, and soy extracts.

An extract can be prepared by drying and subsequently cutting or grinding the dried material. The extraction process may then be performed with the help of an appropriate choice of solvent, typically ethanol/water mixture, methanol, butanol, iso-butanol, acetone, hexane, petroleum ether or other organic solvents by means of maceration, percolation, repercolation, counter-current extraction, turbo-extraction, or by carbon-dioxide hypercritical (temperature/pressure) extraction. The extract may then be further evaporated and thus concentrated to yield by means of air drying, spray drying, vacuum oven drying, fluid-bed drying or freeze-drying, the extract product.

The synthesized compounds can be separated from a reaction mixture and further purified by a method such as column chromatography, high pressure liquid chromatography, or recrystallization. As can be appreciated by the skilled artisan, further methods of synthesizing the compounds herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385;

The compounds of this invention may be modified by appending appropriate functionalities to enhance selective biological properties. Such modifications are known in the art and include those which increase biological penetration into a given biological compartment (e.g., blood, lymphatic system, nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug; or any dosage range in which the low end of the range is any amount between 0.1 mg/day and 400 mg/day and the upper end of the range is any amount between 1 mg/day and 500 mg/day (e.g., 5 mg/day and 95 mg/day, 100 mg/day and 500 mg/day); or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 5 mg/kg/day, 25 mg/kg/day and 75 mg/kg/day) The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound. In one aspect, the dosage in clinical or nutraceutical use is normally within a range of 0.05 g-3 g per adult per day as beta acids.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including glucose level-mediated disorders or symptoms thereof. References which include examples of additional therapeutic agents are: 1) Burger's Medicinal Chemistry & Drug Discovery 6^{th} edition, by Alfred Burger, Donald J. Abraham, ed., Volumes 1 to 6, Wiley Interscience Publication, NY, 2003. Additional therapeutic agents include but are not limited to agents for the treatment of diabetes, glucose regulation, etc.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically and nutraceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The preparations containing beta acids are manufactured by an ordinary method using ordinary recipients and food additives. As an oral preparation, it can be formulated in the form of ordinary tablets, capsules, fine granules or powders. It also can be added to a food product as a pure form or extracted hops. The food product can be a solid, a paste, or a liquid food product, such as milk, tea, soft drinks, juices, coffee, seasonings, cereals, water, cookies, yogurt, chewing gum, chocolate, or soups. The food product can be a “non-alcoholic” food product, that is a food product having low (e.g., <3%, <2%, <1%, <0.5%, <0.25%, <0.1%, <0.05%) or no (e.g., essentially zero) alcohol content. In the invention, the nutraceutical carrier for the compositions herein may include, a base of fruit, vegetables or fruit or vegetable juice or puree, a base of vegetable soup or bouillon, a soya-milk drink, a tea or coffee drink, or a nutritive supplement.

Additionally the components can be fortified with electrolytes, flavors, other plant extracts, preservatives, and other additives, (e.g., vitamin supplements and maltodextrin). Examples of preservatives include, but are not limited to, ascorbic acid and propyl gallate. Examples of electrolytes include, but are not limited to, magnesium sulfate and potassium chloride.

The invention will be further described in the following examples. It should be understood that these examples are for illustrative purposes only and are not to be construed as limiting this invention in any manner.

### Example 1

### (1) Isolation of Alpha-Acids in Hops

Fresh CO₂ hop extract is mixed with three parts water and treated with 20% KOH to reach a pH of about 7.6. Stop mixing and allow the upper beta acids hop oil layer to separate from the lower aqueous layer containing the water soluble alpha acid potassium salt isolate the water layer. Acidify the water layer, the aqueous alkaline solution of alpha acids with sulfuric acid. The alpha acids will oil out and separate from the water layer. The alpha acid layer can be isolated.

### (2) Isolation of Beta Acids

The beta acids can be isolated from the beta acid hop oil layer from above by mixing this fraction with three parts water and adding 20% KOH to reach a pH of about 11.0 to form a water soluble potassium salts of beta acids. Stop mixing, separate the lower water layer from the hop oil layer. The beta acids can be isolated from the water by treating this aqueous alkaline solution with sulfuric acid to “oil” out the beta acids like what was done with the alpha acids.

### (3) Isolation of Xanthohumol

Xanthohumol can be isolated according to a method described in U.S. Pat. No. (3,794,744) issued in 1974.

### (4) Glucose Uptake Enhancing Activity Experiments

### Example 2

### Skeletal Muscle Cells

All skeletal muscle cell strains used are skeletal muscle myoblast cells (purchased from Clonetics) with negative for HIV-I, hepatitis-B & C, mycoplasma, bacteria, yeast and fungi. This Skeletal Muscle Myoblast Cell System contains normal human muscle myoblasts (HSMM). The cell and media System can quickly generate HSMM cultures for the study of cellular development and differentiation, insulin uptake or resistance, or tissue repair. The Skeletal muscle cells were cultured on Cytostar 96-well cultured plate. Cells were labeled with 3H-deoxyglucose (50 μCi/ml, 4 μM) in the presence of 8 mM glucose. Radioactivity was counted on a Microbeta scintillation counter. In beta-acid treated cells (mixture of 65:25:10 of co-Lupulone: lupulone: ad-lupulone, Beta Tec, Washington, D.C.), kinetics of labeled deoxyglucose uptake was directly measured without washing. In order to avoid color quenching of quercetin treated cells, cells were washed with Hanks buffer and then the radioactivity was measured at end-point (18 hr). Error bars show SD (n=3).

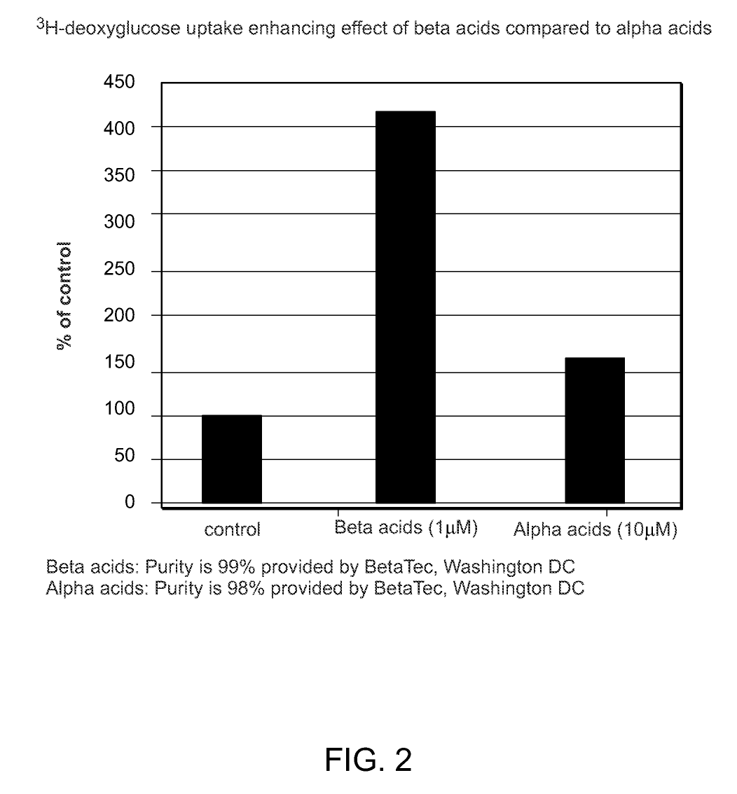

FIG. 2 shows glucose uptake into muscle skeletal cells after 24 hrs incubation in the presence of various hops extracts, such as AH (Alphahop mainly containing alpha acids), xanthohumol that one of the ingredients in hops and BS (Betastab 10A containing 10% beta acids). It is clear that alpha acids and xanthohumol that are representative ingredients in hops have no enhancing effect.

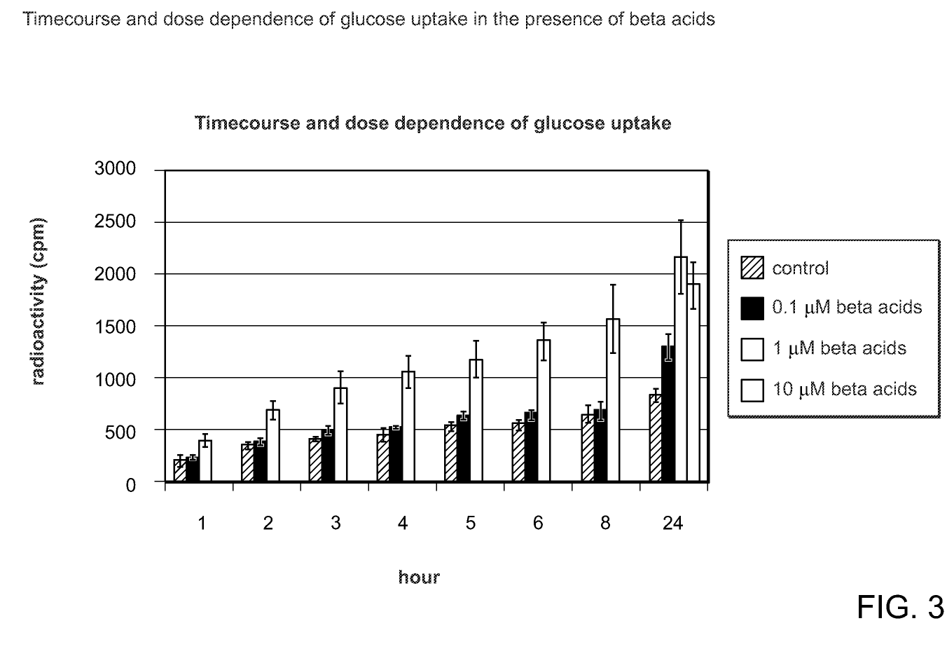

FIG. 3 shows the 3H-deoxyglucose uptake enhancing effect of beta acids compared to pure alpha acids. High purity beta acids showed a strong glucose uptake enhancing effect in the low concentration. Alpha acids are another major ingredient in hops and are known as a naturally occurring antioxidant. They do not show a glucose uptake enhancing effect even at the high concentration. Since beta acids are also categorized as herbal antioxidants, the present enhancing effect is apparently not directly related to the expected anti-oxidation effect.

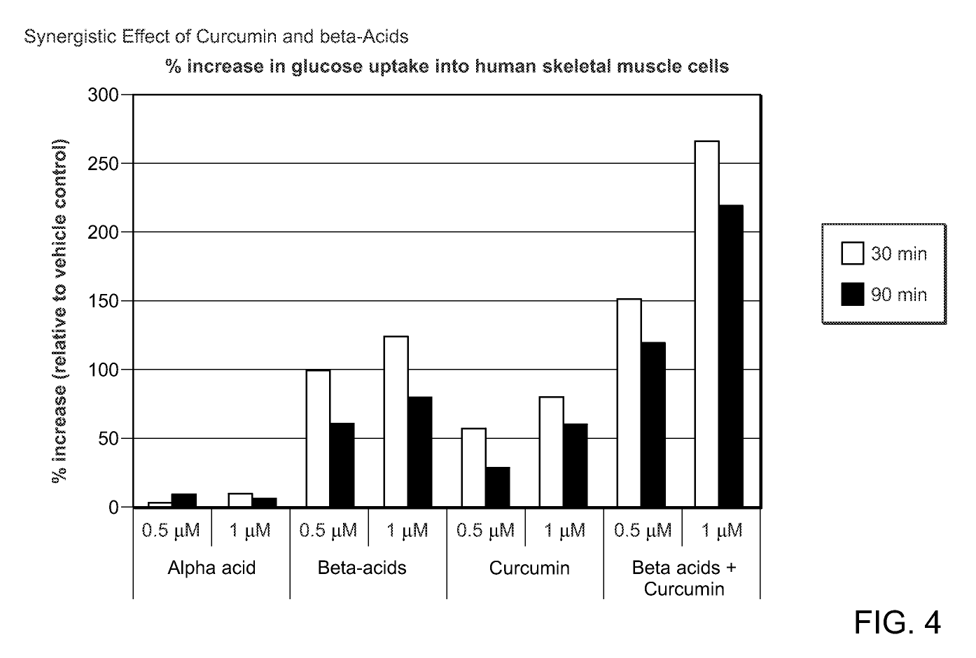

FIG. 4 shows timecourse and dose dependency of glucose uptake in the presence of beta acids. It indicated that this enhancement occurs with dose-dependent manner at least between 0.1 uM and 1 uM. and also the effect was observed higher, as the exposure time was getting longer.

Compounds are prepared in a manner essentially as described above and in the general schemes.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-diabetic composition comprising an effective amount of a hops beta acid or beta acid derivative.

2. The composition of claim 1, further comprising a pharmaceutically acceptable carrier.

3. A nutraceutical composition comprising a hops beta acid or beta acid derivative and a nutraceutically acceptable carrier.

4. A non-alcoholic food product comprising a hops beta acid or beta acid derivative.

5. (canceled)

6. The nutraceutical composition of claim 3, further comprising at least one edible plant extract.

7. The composition of claim 1, further comprising an additional therapeutic agent.

8. (canceled)

9. The composition of claim 1, wherein the hops beta acid derivative is Lupulone, Adlupulone or Colupulone.

10. A method of treating diabetes or a diabetes-associated symptom in a subject in need of such treatment comprising administering to the subject an effective amount of a composition of claim 1.

11. The method of claim 10, wherein the diabetes or diabetes-associated symptom is diabetes mellitus, type-II diabetes, diabetic retinopathy, diabetic neuropathy, or diabetic nephropathy.

12. A method of modulating blood glucose uptake activity in a subject comprising administering to the subject an effective amount of a composition of claim 1.

13. A method for treating diabetes in a subject by enhancing blood glucose uptake, comprising administering to the subject in need of such treatment a pharmaceutical composition comprising an effective amount of hops beta acid.

14. A method for treating obesity in a subject by enhancing blood glucose uptake, comprising administering to the subject in need of such treatment a pharmaceutical composition comprising an effective amount of hops beta acid.

15. A method for stimulating glucose uptake in a subject, comprising administering to the subject in need of such treatment a pharmaceutical composition comprising an effective amount of an active ingredient that is a hops beta acid.

16. The method of claim 15, wherein the composition further comprises a compound found in turmeric.

17. The method of claim 15, wherein the composition further comprises curcumin.

18. The composition of claim 2, further comprising a compound isolated from turmeric.

19. The composition of claim 2, further comprising curcumin.
